(19) United States
(12) Patent Application Publication
(10) Pub. No.: US 2020/0086107 A1
(43) Pub. Date: Mar. 19, 2020

(54) MAGNETICALLY CONNECTED MEDICAL TUBING

(71) Applicant: University of Rochester, Rochester, NY (US)

(72) Inventors: Luis Ignacio RUFFOLO, Rochester, NY (US); Derek S. WAKEMAN, Rochester, NY (US)

(73) Assignee: University of Rochester, Rochester, NY (US)

(21) Appl. No.: 16/569,437

(22) Filed: Sep. 12, 2019

Related U.S. Application Data
(60) Provisional application No. 62/731,038, filed on Sep. 13, 2018.

(51) Int. Cl. A61M 39/10; A61J 15/00; A61M 27/00; A61M 25/00; A61M 16/04

(52) U.S. Cl. CPC A61M 39/10; A61J 15/003; A61J 15/0015; A61J 15/0092; A61M 2205/0288; A61M 25/0017; A61M 16/0465; A61M 2205/0216; A61M 27/00

(57) ABSTRACT

The invention provides systems and methods for reducing injury to patients by dislodgement of implanted or attached medical tubing. The inventive systems and methods provide medical tubing with a releasable magnetic connection. When a stretching force is applied to the medical tubing that would cause the implanted or attached portion to be pulled or separated from the patient, the medical tubing instead separates at the point of the releasable magnetic connection, preventing the force from reaching the portion of the tubing implanted in or attached to the patient, thereby reducing the risk of injury to the patient. The releasable magnetic connection allows the tubing to be easily reconnected once the stretching force on the tubing has been terminated.

### CROSS-REFERENCE TO RELATED APPLICATIONS

This application claims the benefit of and priority to U.S. Provisional Patent Application No. 62/731,038, filed Sep. 13, 2018, the contents of which are incorporated herein by reference for all purposes.

### STATEMENT OF FEDERAL FUNDING

Not applicable.

### BACKGROUND OF THE INVENTION

A variety of medical conditions, such as stroke, inability to swallow, or damage to the esophagus, can render patients unable for extended periods of time to tolerate oral feeding. Doctors typically treat such patients by introducing a gastrostomy tube, or “G tube,” through the abdominal wall into the patient's stomach, allowing the patient to be fed by passing pureed or liquid foods through the G tube into the stomach. Unfortunately, G tubes can be dislodged by being caught on clothing or objects as the patient moves around or, in the case of infants or persons with dementia, because the patient pulls at them. If such dislodgement occurs before a tract has formed around the tube, an emergency room visit is required so the tube can be reinserted. Even after a tract has formed around the G tube, a month or so after its initial placement, dislodgement of the G tube exposes the peritoneal cavity to contamination by inadvertent spillage of gastric contents which can place the patient at risk of infection and even sepsis. Such dislodgement is a frequent cause of the patients or their caretakers seeking medical assistance to have the tube reinserted. From 2016 to 2017, Golisano Children's Hospital, part of the University of Rochester Medical Center, recorded almost 150 emergency department visits due to G tube dysfunction, of which 91 (over 60%) were due to dislodgement. It would be desirable to have G tubes that are less susceptible to being dislodged.

Dislodgement of drainage tubes is also a source of injury to patients. For example, bedridden patients with Foley catheters providing urinary drainage being wheeled to or from their room can have the catheter caught on a door or chair, causing the Foley catheter to be ripped out of their bladder, often with damage to their urethra and, in men, often damage to their prostate gland as well.

Other medical tubing, such as endotracheal tubes and intravenous (“IV”) lines, can also be dislodged. Unplanned extubation of an endotracheal tube, often due to movement of an agitated patient in an ICU, is life-threatening and is one of the metrics measured in tracking ICU quality. It would be desirable to have systems and methods for reducing unplanned dislodgement of endotracheal tubes. The dislocation of IV lines can cause pain. The dislodgement of central venous lines, which are often used to deliver critical life supportive medication such as vasopressors, can result in serious disruption in critical care as well as the risk of exsanguination from large bore dislodgement or hematoma formation. It would be desirable to have additional systems and methods for reducing dislodgement of IV lines, particularly of central venous lines, as well as other medical tubing.

Surprisingly, the inventive tubing systems and methods fulfill these and other needs.

### PARTIES TO JOINT RESEARCH AGREEMENT

Not applicable.

### REFERENCE TO SEQUENCE LISTING OR TABLE SUBMITTED ON COMPACT DISC AND INCORPORATION-BY-REFERENCE OF THE MATERIAL [SPECIFY NUMBER OF DISCS AND FILES ON EACH]

Not applicable.

### SUMMARY OF THE INVENTION

The invention provides systems, devices, and methods for magnetically connected medical tubing. In some embodiments, the invention provides medical tubing system with sections releasably held together by magnetic attraction, comprising (a) a first section of medical grade elastomeric tubing having a length, an exterior, a proximal end and an opposing distal end having a diameter, a first opening at the proximal end and a second opening at the distal end, a first lumen fluidly connected to the first opening, the lumen having a diameter and extending through the first section and fluidly connecting the first opening to the second opening of the first section, (b) a second section of medical grade elastomeric tubing having a length, a proximal end having a first diameter matching the diameter of the distal end of the first section, an exterior, a proximal end having a first opening and a distal end having a second opening, which distal end further optionally (i) has a second diameter greater than the diameter, or (ii) is adapted to expand in diameter from that of the first diameter to a larger diameter, a first lumen of the second section, the first lumen of the second section having a diameter extending through the second section and fluidly connecting the first opening to the second opening of the second section, and (c) the first and the second sections are releasably held together by a selected magnetic force between (i) at least one magnet disposed on the exterior of the distal end of the first section and at least one magnet disposed on the exterior of the proximal end of the second section, the at least one magnet on the first and second sections positioned to releasably hold the second opening of the first section and the first opening of the second section together with a first selected magnetic force, (ii) at least one magnet disposed on the exterior of the distal end of the first section and a ferromagnetic material disposed on the exterior of the proximal end of the second section, the at least one magnet and the ferromagnetic material positioned to releasably hold second opening of the first section and the first opening of the second section of tubing together with a first selected magnetic force, or (iii) a ferromagnetic material disposed on the exterior of the distal end of the first section and at least one magnet disposed on the exterior of the proximal end of the second section, the ferromagnetic material and the at least one magnet positioned to releasably hold the second opening of the first section and the first opening of the second section together with a first selected magnetic force. In some embodiments, one or more additional magnets are disposed around the exterior of the distal end of the first section, around the exterior of the proximal end of the second section, or both. In some embodiments, (i) the at least one magnet of element (c)(i) disposed on the distal end of the first section is configured as a ring or a cylinder around the exterior of the distal end of the first section and is attached to the exterior, and the at least one magnet of element (c)(i) disposed on the proximal end of the second section of element (c)(i) is configured as a ring or cylinder around the proximal end of the second section and is attached to the exterior, (ii) the at least one magnet of element (c)(ii) is configured as a ring or cylinder around the exterior of the distal end of the first section and is attached to the exterior, and the ferromagnetic material magnet of element (c)(i) is configured as a ring or cylinder around the exterior of the proximal end of the second section and is attached to the exterior, or (iii) the ferromagnetic material of element (c)(iii) is configured as a ring or cylinder around the exterior of the distal end of the first section and is attached to the exterior, and the at least one magnet of element (c)(iii) is configured as a ring or cylinder around the exterior of the proximal end of the second section and is attached to the exterior. In some embodiments, the attachment of the magnets and the ferromagnetic material is by a medical grade adhesive. In some embodiments, the one least one magnet on the first section or on the second section, or both, and the ferromagnetic material, is covered with, or embedded in, a medical grade elastomeric material. In some embodiments, the distal end of the first section terminates into a nozzle fitting into the first lumen of the second section. In some embodiments, the nozzle is cylindrical. In some embodiments, the nozzle has a taper. In some embodiments, the proximal end of the second section contains a one-way valve, which valve is held open when the nozzle of the first section is present and which is in closed position when the nozzle is not present. In some embodiments, the distal end of the second section has a diameter expanded from that of the first diameter of the second section or has an adaption to increase in diameter when desired. In some embodiments, the adaption to increase in diameter when desired is an inflatable balloon. In some embodiments, the first and the second sections comprise a second lumen extending therethrough fluidly connecting an inflation port at the proximal end of the first section with the inflatable balloon on the distal section of the second section. In some embodiments, the adaption to increase in diameter when desired is an expandable bolster. In some embodiments, the expanded diameter of the distal end of the second section is created by two or more wings extending from the distal end. In some embodiments, the expanded diameter of the distal end of the second section is a rim or lip of material with a diameter larger than that of the second section. In some embodiments, the expanded diameter of the distal end of the second section is an expanded tip. In some embodiments, the expanded diameter of the distal end of the second section is bulbous. In some embodiments, the expanded diameter of the distal end of the second section is a hemispherical dome having a wide bottom, which wide bottom is disposed facing towards the first section of the medical tubing. In some embodiments, the first section is longer than the second section. In some embodiments, the second section has an external retention ring positioned between the connection of the first and second sections and the distal end of the second section. In some embodiments, the medical tubing is a gastrostomy (“G”) tube, a jejunostomy (“J”) tube, or a G-J tube. In some embodiments, the distal end of the medical tubing is adapted to mate to a low-profile button, with the lumen of the medical tubing system fluidly connected to a lumen in the low-profile button and further fluidly connecting to a tube extending distally from the low-profile button. In some embodiments, the medical tubing is a G tube, a J tube, or a G-J tube. In some embodiments, the medical tubing is a drainage catheter. In some embodiments, the catheter is a Foley catheter. In some embodiments, the medical tubing is a chest tube. In some embodiments, the medical tubing is an intravenous line. In some embodiments, the intravenous line is a central line or an arterial line. In some embodiments, the medical tubing is an endotracheal tube. In some embodiments, the preselected magnetic force is between 2 pounds and about 30 pounds. In some embodiments, the preselected magnetic force is between 5 pounds and about 20 pounds. In some embodiments, the preselected magnetic force is between 8 pounds and about 15 pounds. In some embodiments, the preselected magnetic force is between about 8 pounds and about 12 pounds. In some embodiments, the preselected magnetic force is about 30 pounds. In some embodiments, the magnet, the ferromagnetic material, or both, are removable. In some embodiments, the distal end of the first section and the proximal end of the second section are configured to reversibly hold the magnets of element (i) or the magnets and ferromagnetic materials of elements (ii) or (iii), respectively. In some embodiments, the configuration of the ends is by (i) providing the distal end of the first section with male or female helical ridges and providing a magnet or a ferromagnetic material, or a holder holding the magnet or the ferromagnetic material with helical ridges mating to the helical ridges of the distal end of the first section, and (ii) providing the proximal end of the second section with male or female helical ridges and providing a magnet or a ferromagnetic material, or a holder holding the magnet or the ferromagnetic material with helical ridges mating to the helical ridges of the proximal end of the second section.

In another group of embodiments, the invention provides medical tubing systems with a low-profile button, comprising (a) a first section of medical grade elastomeric tubing having a length, a diameter, an exterior, a proximal end and an opposing distal end, a first opening port at the proximal end, a first lumen fluidly connected to the first opening port, the lumen having a diameter, and extending through the length of the first section from the first opening to a second opening in the distal end of the first section, (b) a low-profile gastrostomy button having a proximal, outward facing side and a distal side, the button further having a first opening sized to accept the distal end of the first section, and a lumen of the first opening fluidly connected to a lumen of tubing extending distally from the button to a second opening, the tubing having a first diameter, and, (c) which the second opening in the distal end of the first section is releasably held in the first opening of the button by magnetic force created by (i) at least one magnet disposed on the exterior of the distal end of the first section and at least one magnet disposed on the exterior of the proximal, outward facing side of the button, (ii) at least one magnet disposed on the exterior of the distal end of the first section and a ferromagnetic material disposed on the exterior of the proximal, outward facing side of the button, or (iii) a ferromagnetic material disposed on the exterior of the distal end of the first section and at least one magnet disposed on the exterior of the proximal, outward facing side of the button. In some embodiments, the at least one magnet disposed on the distal end of the first section of element (c)(i) is configured as a ring or cylinder around the exterior of the first section, and the at least one magnet disposed on the button is configured as a ring or cylinder surrounding the first opening of the button. In some embodiments, the at least one magnet disposed on the distal end of the first section of element (c)(ii) is configured as a ring or cylinder around the exterior of the first section, and the ferromagnetic material disposed on the button is configured as a ring or cylinder surrounding the first opening of the button. In some embodiments, the ferromagnetic material disposed on the distal end of the first section of element (c)(iii) is configured as a ring or cylinder around the exterior of the first section, and the at least one magnet disposed on the button is configured as a ring or cylinder surrounding the first opening of the button. In some embodiments, the at least one magnet on the first section, the at least one magnet on the button, or the ferromagnetic material, is coated with a medical grade elastomeric material. In some embodiments, the distal end of the first section terminates in a nozzle which fits into the first opening of the button. In some embodiments, the first opening of the button comprises a one-way valve, which valve is held open when the nozzle of the first section is present and which is in closed position when the nozzle is not present. In some embodiments, the distal end of the tubing extending distally from the button has a second diameter larger than that of the first diameter of the tubing extending distally from the tubing, or has an adaption to increase to the second diameter when desired. In some embodiments, the adaption to increase to the second diameter when desired is an inflatable balloon. In some embodiments, the button comprises an inflation port and a second lumen fluidly connected to the inflation port and to the inflatable balloon on the distal section of the tubing extending distally from the button. In some embodiments, the adaption to increase in diameter when desired is an expandable bolster. In some embodiments, the expanded diameter of the distal end of the tubing extending distally from the button is created by two or more wings extending from the distal end, a rim or lip of material with a diameter larger than that of the tubing extending distally from the button, an expanded tip, or a hemispherical dome having a wide bottom, which wide bottom is disposed facing towards the button. In some embodiments, the button is configured to reversibly hold the magnet of element (i) or the ferromagnetic material of element (ii). In some embodiments, the configuration of the button is by providing the button with male or female helical ridges surrounding the button around the first opening and by providing the magnet, the ferromagnetic material, or a holder holding the magnet or the ferromagnetic material with helical ridges mating to those surrounding the button. In some embodiments, the system further comprises a bridging tube configured to screw into and connect the first and second sections when the magnets or the magnets and ferromagnetic material of the holder holding the magnet or the ferromagnetic material are not in position in the tubing system. In some embodiments, the medical tubing is a gastrostomy (“G”) tube, a jejunostomy (“J”) tube, or a G-J tube.

In yet another group of embodiments, the invention provides methods of reducing injury to a patient from accidental dislodgement of medical tubing having a first diameter, a lumen and an end implanted in the patient and having a first opening, and connected to medical tubing extending externally from the patient and having a lumen in fluid connection with the first opening, wherein the medical tubing has a first force required to dislodge the medical tubing implanted in the patient, the methods comprising: providing a releasably sealable break in the medical tubing extending externally from the patient, the break creating a first end of a first section of tubing proximal to, but with a portion external to, the patient, and having an exterior and a lumen, and a first end of a second section, which second section is distal to the patient compared to the first section, and has an exterior and a lumen, the first ends of the first and second sections being releasably held together, with the lumens in fluid connection, by force of magnetic attraction between (i) at least one magnet disposed on the exterior of the first section and at least one magnet disposed on the exterior of the second section, (ii) at least one magnet disposed on the exterior of the first section and a ferromagnetic material disposed on the exterior of the second section, or (iii) ferromagnetic material disposed on the exterior of the first section and at least one magnet disposed on the exterior of the second section, wherein the force of magnetic attraction is less than the first force required to dislodge the medical tubing implanted in the patient. In some embodiments, one or more additional magnets are disposed around the exterior of the first end of the first section, around the exterior of the first end of the second section, or around both. In some embodiments, either (i) the at least one magnet disposed on the first end of the first section is configured to surround the exterior of the first end of the first section and is attached to the exterior, and the at least one magnet disposed on the first end of the second section or the ferromagnetic material disposed on the first end of the second section is configured to surround the first end of the second section and is attached to the exterior, or, (ii) the ferromagnetic material disposed on the first end of the first section is configured to surround the exterior of the first end of the first section and is attached to the exterior, and the at least one magnet disposed on the first end of the second section is configured to surround the exterior of the first end of the second section and is attached to the exterior. In some embodiments, the at least one magnet of element (i), or the ferromagnetic material of element (ii), or both, is configured as a ring or a cylinder. In some embodiments, the attachment of the magnets or of the ferromagnetic material, or of both, to the exteriors is by a medical grade adhesive. In some embodiments, the attachment of the at least one magnet to the exterior of the first end of the first section and of the at least one magnet or the ferromagnetic material disposed on the exterior of the first end of the second section in element (i) is reversible. In some embodiments, the reversible attachment is by screwing together helical ridges on the magnet or the ferromagnetic material, or a holder holding the magnet or the ferromagnetic material, to mated helical ridges on the exterior of the first end of the first section or of the exterior of the first end of the second section in element (i), respectively. In some embodiments, (i) the ferromagnetic material configured to surround the exterior of the first end of the first section is attached to the exterior by screwing together helical ridges provided on the ferromagnetic material or a holder holding the ferromagnetic material to mating helical ridges on the exterior, and (ii) the at least one magnet disposed on the end of the second section is attached to the exterior of the first end of the second section by screwing together helical ridges on the magnet or a holder holding the magnet to mating helical ridges on the exterior. In some embodiments, the one least one magnet on the first section or on the second section, or both, and the ferromagnetic material, is coated by, or embedded in, a medical grade elastomeric material. In some embodiments, the first end of the second section terminates in a nozzle fitting into the lumen of the first end of the first section. In some embodiments, the nozzle is cylindrical. In some embodiments, the nozzle has a taper. In some embodiments, the first end of the first section contains a one-way valve, which valve is held open when the nozzle of the second section is present and which is in closed position when the nozzle is not present. In some embodiments, the end of the medical tubing implanted in the patient has a second diameter expanded from that of the first diameter of the medical tubing or has an adaption to increase the first diameter to a second diameter when desired. In some of these embodiments, the adaption to increase in diameter when desired is an inflatable balloon. In some embodiments, the first and the second sections comprise a second lumen extending therethrough fluidly connecting an inflation port external to the patient with the inflatable balloon on the end of the medical tubing implanted in the patient. In some embodiments, the adaption to increase in diameter when desired is a bolster. In some embodiments, the expanded diameter of the distal end of the second section is created by two or more wings extending from the distal end, a rim or lip of material with a diameter larger than that of the second section, an expanded tip, a bulbous tip, or a hemispherical dome having a wide bottom, which wide bottom is disposed facing towards the first section of the medical tubing. In some embodiments, the second section is longer than the first section. In some embodiments, the first section has an external retention ring positioned between the break between the first and second sections and the patient. In some embodiments, the medical tubing is a gastrostomy (“G”) tube, a jejunostomy (“J”) tube, or a G-J tube. In some embodiments, the medical tubing is a drainage catheter. In some embodiments, the catheter is a Foley catheter. In some embodiments, the medical tubing is a chest tube. In some embodiments, the medical tubing is an intravenous line. In some embodiments, the intravenous line is a central line. In some embodiments, the medical tubing is an endotracheal tube. In some embodiments, the end of the medical tubing implanted in the patient terminates in a low-profile button, and the medical tubing external to the patient but connected to the medical tubing implanted in the patient is connected to the medical tubing implanted in the patient by mating the lumen of the medical tubing external to the patient to a lumen in the low-profile button, which lumen in the button is fluidly connected to a lumen extending from the low-profile button to the end implanted in the patient. In some embodiments, the medical tubing is a G tube, a J tube, or a G-J tube. In some embodiments, the force of magnetic attraction is between 1 pound and about 30 pounds. In some embodiments, the force of magnetic attraction is between about 5 pounds and about 20 pounds. In some embodiments, the force of magnetic attraction is between about 8 pounds and about 15 pounds. In some embodiments, the force of magnetic attraction is between about 8 pounds and about 12 pounds.

In a further group of embodiments, the invention provides methods for reducing injury to a patient from accidental dislodgement of medical tubing having a first diameter, a lumen and an end implanted in the patient and having a first opening, and extending to a low-profile button disposed on an exterior surface of the patient, the button having a lumen therein and having an exterior surface defining a second opening of the lumen, wherein the lumen is fluidly connected through the lumen of the medical tubing from the first opening to the second opening, wherein the second opening is fluidly connected to a lumen of medical tubing extending externally from the patient, which medical tubing extending externally from the patient has an exterior, wherein the medical tubing has a first force required to dislodge the medical tubing implanted in the patient, the method comprising: providing a releasable magnetic connection between exterior surface defining the lumen of the button and the medical tubing extending externally from the patient, the releasable magnetic connection being formed by force of magnetic attraction between (i) at least one magnet disposed on the exterior surface of the button and at least one magnet disposed on the exterior of the medical tubing extending externally from the patient, (ii) at least one magnet disposed on the exterior surface defining the lumen of the button and ferromagnetic material disposed on the exterior of the medical tubing extending externally from the patient, or (iii) ferromagnetic material disposed on the exterior surface defining the lumen of the button and at least one magnet disposed on the exterior of the medical tubing extending externally from the patient, wherein the force of magnetic attraction is less than the first force required to dislodge the medical tubing implanted in the patient. In some embodiments, the one or more additional magnets are disposed around the exterior surface defining the lumen of the button or the exterior of the medical tubing extending externally from the patient, or both. In some embodiments, either (i) the exterior surface defining the lumen of the button is configured in a ring and the at least one magnet or the ferromagnetic material disposed on the exterior surface defining the lumen of the button is configured as a ring or cylinder surrounding the lumen of the button and is attached to the exterior surface defining the lumen of the button, or (ii) the exterior of the medical tubing extending externally from the patient is cylindrical and the at least one magnet or the ferromagnetic material disposed on the exterior of the medical tubing is configured as a ring or cylinder and is attached to the exterior surface. In some embodiments, the attachment of the at least one magnet to the exterior surface and the attachment of the ferromagnetic material to the exterior surface is by medical grade adhesive. In some embodiments, the attachment of the at least one magnet to the exterior surface and the attachment of the ferromagnetic material to the exterior surface is by screwing together helical ridges on the at least one magnet or the ferromagnetic material, or on a holder holding the at least one magnet or the ferromagnetic material, to mating helical ridges in the exterior surface defining the lumen of the button. In some embodiments, the medical tubing is a gastrostomy (“G”) tube, jejunostomy (“J”) tube, or a G-J tube. In some embodiments, the force of magnetic attraction is between 1 pound and about 30 pounds. In some embodiments, the force of magnetic attraction is between about 5 pounds and about 20 pounds. In some embodiments, the force of magnetic attraction is between about 8 pounds and about 15 pounds. In some embodiments, the force of magnetic attraction is between about 8 pounds and about 12 pounds.

### BRIEF DESCRIPTION OF THE DRAWINGS

FIG. 1. FIG. 1 is a drawing of a conventional gastrostomy tube modified by a releasable, “in-line” magnetic connection. Gastrostomy tube 100 is divided into two sections, 101 and 102, with a break 103 between them, which break is releasably closed by the force created by magnetic attraction between first magnet 104 and second magnet 105, which surround tube 100 and in position to attract one another. When the break is releasably held closed by the attraction of magnets 104 and 105, food introduced into open end 106 of tube 100 then traverse the tube to a second opening 107, in distal end 108. Gastrostomy tube 100 may have an external retention ring 108 to keep tube 100 from being inadvertently pushed too deep into the patient when in position, and may have a balloon port 109 fluidly connected to a lumen (not shown) in tube 100 allowing inflation of a retention balloon (not shown).

FIGS. 2A and 2B. FIG. 2A. FIG. 2A is a drawing showing an extension tube configured with an “in-line” releasable magnetic connection, for use with a low-profile “button” gastrostomy tube system. Extension tube 100 is divided into two sections, 101 and 102. Section 101 has a first opening (sometimes called a “food port”) 104 for introducing food into the lumen of tube 100. First section 102 has two ends, 103 and 107. Second end 104 has a second opening 105, which is surrounded by first magnet 106. Second section 102 has a first end, 107, surrounded by second magnet 108. Tube second section 102 terminates in second end 109, from which extends nozzle 110. Nozzle 110 is configured to fit into a low-profile gastrostomy button. In some embodiments, second end 109 has a magnet or a ferromagnetic material disposed, for example, around nozzle 110, to releasably hold nozzle 110 to the lumen of a low-profile gastrostomy button. FIG. 2B. FIG. 2B is a drawing showing a low-profile gastrostomy button 120, which has been modified by addition of magnet 121 surrounding first opening 122. First opening 122 connects to a lumen (not shown) in tube 124, which extends distally to second opening 125. Tube 124 is surrounded on its distal end by balloon 126, which has been inflated in this view by water introduced through balloon port 123 and flowing through a second lumen (not shown) in tube 124. First opening 122 can be protected from introduction of unwanted substances when not in use by closure 127.

### DETAILED DESCRIPTION

Medical tubing is often implanted into patients. Some medical tubing, such as feeding tubes and Foley catheters, have adaptions to retain the tubing in the desired position inside the patient. While such retention adaptations are normally useful, they can cause pain or even injury to the patient if the portion of the tubing outside the patient is inadvertently caught on an object and pulls the internal portion of the tubing out of the patient. For example, Foley catheters, which have an inflatable balloon to hold the catheter in a patient's bladder, also have an external section of tubing connecting to a urine collection bag. The external tubing section of a Foley catheter can catch on objects as the catheterized patient is being moved, suddenly jerking the catheter from the patient's bladder and causing injury to the patient's urethra (and, if the patient is a male, to the patient's prostate gland as well). Thus, reducing dislodgement of drainage catheters implanted in patients would reduce the risk of patient discomfort and injury.

Similarly, feeding tubes placed through the abdomen into the stomach (gastrostomy, or “G” tubes), the jejunum (“J” tubes), or both the stomach and the jejunum (“G-J tubes”), typically have an internal retention adaptation, such as an inflatable balloon, and can likewise be dislodged by being caught on objects around the patient or, in some cases, by being pulled on by the patient. Dislodgement of a G, J, or G-J tube in the first 24 hours is a surgical emergency, and dislodgement before a tract has formed around the tube typically requires medical intervention. Once a tract has formed around the tube, typically by six weeks after the tube has been placed, dislodgement is a nuisance rather than an emergency, but may still require medical personnel to reinsert the tube. Thus, reducing the number of dislodgements of such tubes can significantly reduce risk to the patient and the burden dislodgements place on the medical system.

Other medical tubing, such as intravenous (IV) lines, can also have sections which are implanted and which can cause pain, injury, or both, if dislodged. This is a particular concern for central lines (lines into central veins) and arterial lines, which are typically placed to measure blood pressure in very sick patients. Such lines are often sutured to the patient to reduce the risk of dislodgement, but with the consequence that any force on the line that is greater than the strength of the sutures tears the patient's skin and causes both injury and, if the patient is conscious, pain.

Surprisingly, the invention provides systems and methods to reduce the long-felt problem of inadvertent dislodgment of feeding tubes, drainage tubes, sutured IV lines, and of other forms of medical tubing implanted in patients and having an external portion which can cause inadvertent dislodgement of the implanted tubing. In embodiments of the inventive systems and methods, a releasable, magnetic connection is formed between portions of the tubing external to the patient. The size and strength of the magnet or magnets creating the releasable, magnetic connection (either to an opposing magnet or to a piece of ferromagnetic material) are selected so that the force of the magnetic attraction holding the connection together is less than the force expected to be needed to dislodge the implanted portion of the tubing from the patient or, in the case of sutured lines, to tear the lines from the patient. Application of a tension, or stretching, force to the tubing with a force that would other be sufficient to dislodge the internal tubing breaks the magnetic connection, reducing the chance that enough force will be transmitted to the implanted portion of the tubing to dislodge it (or, in the case of sutured lines, that enough force will reach the sutures to break them or to tear the skin to which the sutures are attached). Once the cause of the stretching force has been determined and reduced or eliminated, for example, by removing the tubing from an object on which the tubing has caught, the releasable magnetic connection can be readily reestablished.

In some embodiments of the invention, the releasable, magnetic connection is created in medical tubing in which one section is implanted in a patient by providing medical tubing which has two ends external to the patient, which ends are releasably held together by magnetic attraction between magnets, or between one or more magnets on one end and a ferromagnetic material on the other end. The magnetic attraction holding these two ends together is selected (as discussed further below) to be less than the force expected to be required to pull the implanted portion of the tubing out of the patient. In some preferred embodiments, one end of the releasable magnetic connection has a nozzle which fits into the lumen of the tubing on the other side of the connection when the two ends are held together by the magnetic connection. Such a nozzle has several advantages, including reducing the potential for leakage at the connection point. Embodiments in which medical tubing is provided in which two ends of the tubing are held together with a releasable magnetic closure are sometimes referred to herein as “in-line” embodiments.

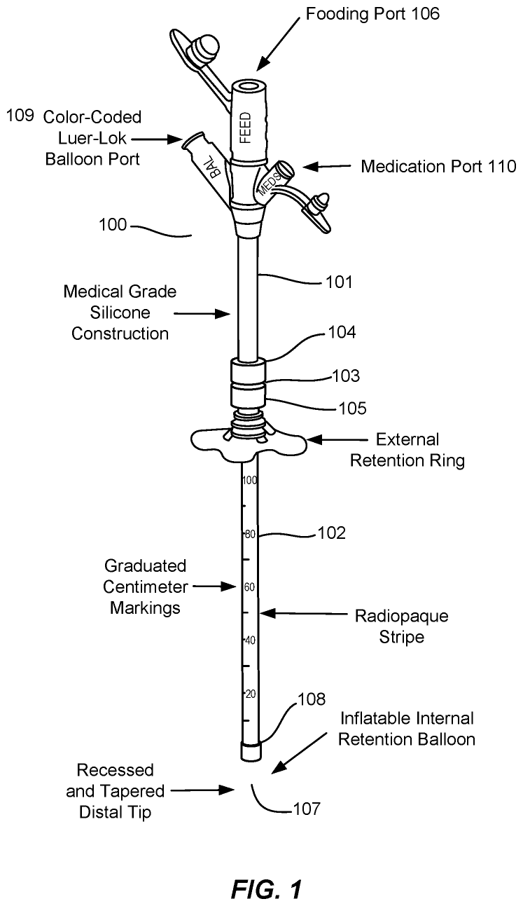

Some features of such “in-line” embodiments may be better illustrated by reference to FIG. 1. FIG. 1 shows a depiction of a commercially available multi-lumen G tube, 100, which has been modified to provide a releasable magnetic connection. G tube 100 has been provided with a break, 103, in tube 101, and two magnets, 104 and 105, disposed around tube 101, which releasably connect tube 101 at break 103. Break 103 is positioned near external retention ring 108. External retention ring 108 has been positioned just before the position on tube 100 intended to be within the patient's abdomen. Section 102 of the tube is the portion intended to be implanted within the patient. In use, a tension, or stretching, force on tube 101 distal to the patient will sever the magnetic connection at break 103 before the force reaches section 102.

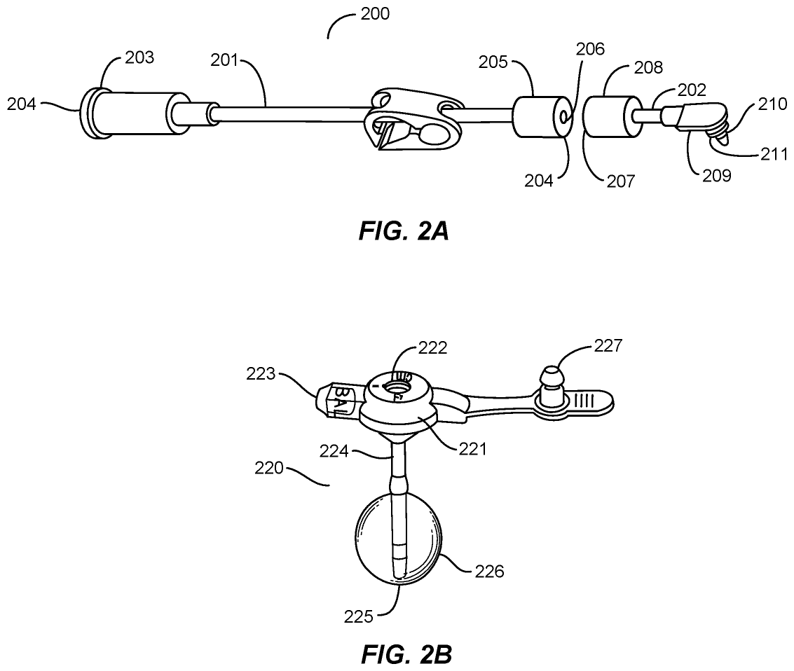

An expanded view of an in-line embodiment having a releasable magnet connection in in-line embodiments of the inventive systems and methods may be seen by reference to FIG. 2A. FIG. 2A is an illustration of a conventional extension tube 200 for use with a conventional low-profile gastrostomy “button” (the button is not shown in FIG. 2A). Tube 200 has been modified from the conventional tube by being provided with a break, creating new ends 204 and 207. Ends 204 and 207 are releasably held together by placing magnets 205 and 208 sufficiently close to the ends that the magnets chosen can hold ends 204 and 207 together with the desired amount of magnetic force, reconnecting lumen 206 of tube 200 in fluid communication with end 209. End 209 has a nozzle 210 projecting therefrom, which nozzle 210 is configured to mate with a low-profile gastrostomy button, such as the one depicted in FIG. 2B. Systems employing an extension tube mating to a low-profile button are sometimes referred to herein as “tube-button systems.” Low-profile gastronomy buttons (sometimes referred to as “G-buttons”) are common features of conventional gastronomy tubes and are, accordingly, well known to persons of skill.

FIG. 2B is an illustration of a portion of an alternative embodiment of the inventive systems and methods. FIG. 2B shows a low-profile gastrostomy button 220, which has been modified from a standard button by placement of magnet 221 around the button's lumen, 222. Magnet 221 is positioned so that it can form a releasable magnetic connection with a magnet disposed on end 209 or disposed around nozzle 210 of gastrostomy tube 200 shown in FIG. 2A, or with a ferromagnetic material disposed in one of those locations.

Conventional feeding tubes configured to be used with a low-profile button typically lock to the button by, for example, aligning a tooth on the nozzle with a slot in the button, inserting the nozzle so that the tooth enters the slot, and then rotating the nozzle so that the tooth rotates away from the slot, keeping the nozzle from detaching from the button unless the nozzle is rotated back to realign the tooth with the slot. If such a locking system is used in a system that also has a releasable magnetic connection between the tube and the button, it is preferably not used at the same time as the releasable magnetic connection, as the locking of the tube to the button would prevent the feeding tube from releasably disconnecting from the button when a tension, or stretching, force reaches the tube adjacent to the side of the button external to the patient.

Releasable magnetic connections such as that described above can be also introduced in drainage catheters or vascular lines to reduce or eliminate dislodgement of the catheter or line due to excess stretching force on the catheter or line. As with the releasable magnetic connections in feeding tubes described above, releasable magnetic connections in the catheters or lines can be created by disposing magnets on both sides of the connection, or by a combination of one or more magnets on one side and ferromagnetic material on the other side of the connection to provide the desired force of magnetic attraction.

The application of a stretching force sufficient to break the force of the magnetic attraction holding the two ends of the releasable magnetic connections together causes the ends of the tubing on either side of the releasable magnetic connection to separate. This is the intended result, as it reduces the potential for dislodgement of the implanted tubing from the patient or, in the case of sutured vascular lines, dislodging the lines, tearing the patient's skin, or both. The two disconnected ends are preferably then wiped with an alcohol swab or similar antiseptic agent to remove any foreign material that may have contacted the ends, and the ends are then placed together to reform the releasable magnetic connection between them. In some embodiments, the exterior of the tubing on either side of the break has a visible line to guide the practitioner in reconnecting the ends without introducing a twist into the tubing.

IV tubing for saline infusions and the like are typically hung from wheeled IV poles, which patients can wheel alongside themselves as they walk or roll a wheelchair along a hospital corridor. Such IV lines are typically secured to the patient with tape. If a releasable magnetic connection is provided in the line in which the force of the magnetic attraction of the releasable magnetic connection is less than the force needed to pull the taped IV line from the patient, the releasable magnetic connection can be placed anywhere along the line between the connection of the IV line to the bag on the IV stand and the tape holding the IV line on the patient, as the releasable magnetic connection will release the line without harm to the patient if anywhere along the line, the line catches on something. Positioning of the releasable magnetic connection closer to the patient is, however, generally desirable as it facilitates ease of reforming the releasable magnetic connection by someone attending to the patient. Central venous lines desirably have a nozzle on the side of the releasable magnetic connection distal to the patient, which opens a one-way valve on the side of the releasable magnetic connection proximal to the patient when the nozzle is present. Upon application of a force that causes the releasable magnetic connection to disconnect, the removal of the nozzle causes the one-way valve to close, preventing loss of blood from the tubing and, more importantly, from the patient. Such nozzles may have a taper in the direction in which they insert into the valve.

In preferred embodiments, the releasable magnetic connection is disposed close to where the medical tubing is secured to the patient or enters the patient. This is particularly important for tubing, such as endotracheal tubes, which are often dislodged by the arm or body movement of an agitated or cognitively impaired patient. On the other hand, the area around the releasable magnetic connection may be somewhat less flexible than other parts of the tubing and it may be more convenient for doctors or nurses securing the tubing that the releasable magnetic connection be an inch or more away from the site at which the tubing is secured to the patient or, for tubing that is not secured, at which it enters the patient. Accordingly, it is generally desirable that the releasable connection in “in-line” embodiments of the inventive systems be disposed between about 1 inch to about 12 inches from the point at which the tubing is secured to the patient or, for tubing that is not secured, enters the patient's skin or connects to a button, in some embodiments is about 2 to about 10 inches, in some embodiments is about 2 to about 8 inches, and more preferably is about 2 to about 6 inches from the point at which the tubing is secured to the patient or, for tubing that is not secured, enters the patient's skin, where “about” for purposes of this measurement means ±0.5 inch.

### Magnets and Ferromagnetic Materials

The inventive systems and methods employ combinations of magnets or of magnets and ferromagnetic materials to provide releasable magnetic connections in medical tubing. As is well known, magnets have two poles, and two magnets disposed with opposite poles facing each other will be attracted to each other. The releasable magnetic connection will generally be understood to have two opposing sides which, when placed in contact with other, are releasably held together by a magnetic force created by the presence at the junction of the two sides of either (a) one or more magnets on the first side being attracted to one or more magnets on the second side or (b) one or more pieces of ferromagnetic material on the first side being attracted to one or more magnets on the second side. A releasable magnetic connection connected by magnets facing each other on the two sides of the connection will be understood to have the magnets oriented so as to attract each other. Ferromagnetic materials are generally defined as materials that are strongly attracted to a magnet. If the releasable magnetic connection is created by a combination of one or more magnets and one or more pieces of ferromagnetic material, it is understood that the magnet(s) and one or more pieces of ferromagnetic will be disposed on opposing sides of the connection in positions such that the magnet on one side of the connection is attracting a facing piece of ferromagnetic material on the other side of the connection. The one or more pieces of ferromagnetic material will generally be sized, shaped, and positioned to match the size, shape and position of the magnet(s) with which the pieces of ferromagnetic material will magnetically attach when the two sides of the releasable magnetic connection are brought into contact to form the connection.

The properties of both magnets and of ferromagnetic materials are well known and both are commercially available. While electromagnets can be used, permanent magnets are preferred for use in the inventive systems and methods, as they avoid the need for providing a source of electricity and wiring to the magnets. Permanent magnets are typically classified based on the materials of which they are made: ferrites; alnicos (aluminum-nickel-cobalt alloy); ceramics; samarium-cobalts; and neodymium alloy (neodymium-iron-boron, also referred to as “NIB” or “NdFeB”). Magnets made of any of these materials can be used so long as they have the desired magnetic strength when forming a magnetic connection along the long axis of the tubing, at sizes suitable to be attached to the medical tubing with which the magnets are contemplated for use.

In some preferred embodiments, the magnets are of NIB, as neodymium magnets are the strongest permanent magnets available. Although these magnets are made of an alloy, they are commonly referred to as “neodymium” magnets, and references herein to “neodymium magnets” will be understood to refer to magnets made of NIB alloy unless otherwise required by context. Neodymium is subject to corroding in the presence of moisture, so neodymium magnets used in embodiments of the present invention are preferably coated to prevent corrosion in use, which might affect their binding to another magnet or to ferromagnetic material used to create a releasable magnetic connection. The coating is preferably a coating approved for use in medical applications. In some embodiments, the magnets may be coated with or embedded in, the same elastomeric material as that forming the medical tubing to be connected by the magnets. If a thick coating is used, the magnets or magnet and ferromagnetic material will be separated by the additional distance of the thickness of the coating. That additional thickness should be taken into account when calculating the magnetic force (“pull force”) between the magnets forming the magnetic connection, or of the magnets and ferromagnetic material forming the magnetic connection, to verify that the magnets, or magnets and ferromagnetic material will form a magnetic connection releasably holding the tubing together with the desired magnetic force. Methods of testing of the pull force of magnets are well known and can be accomplished by, for example, using a calibrated scale and a ferrous test piece. Kits for performing magnetic pull tests are commercially available from, for example, Industrial Magnetics, Inc. (Boyne City, Mich.).

Suppliers such as K&M Magnetics, Inc. (Pipersville, Pa.) and Apex Magnets (Petersburg, W. Va.) sell neodymium magnets in dozens of sizes of rings or cylinders with central lumens of various sizes, all with axial magnetism, and of pull strengths ranging from a little over 1 pound to dozens of pounds. The strength of any particular magnet can effectively be doubled by stacking it with another magnet, so if the pull strength desired for use in the inventive systems and methods happens to be greater than that of individual magnets that are commercially available in a desired size or configuration, the practitioner can simply stack together two (or more) magnets of the desired size or configuration to create a magnet that will provide the desired pull strength. Neodymium magnets become demagnetized if heated over 80° C.; accordingly, neodymium magnets used in the inventive systems and methods are preferably not autoclaved or otherwise subjected to temperatures 80° C. or higher and preferably not to temperatures over 70° C. Coatings covering the magnets are preferably cleaned by wiping them with alcohol swabs or with other antiseptic agents acceptable in a medical environment.

Ferromagnetic materials are materials that are attracted to magnets, and include iron, nickel, cobalt, most of their alloys, and some compounds of rare earth metals. Such materials, and items made from them, are well known.

### Placement of the Magnets and Ferromagnetic Material

In some embodiments, one or a plurality of individual magnets can be disposed around the exterior of the medical tubing at the point forming the releasable connection. For example, a small magnet can be attached at one point and another small magnet attached 180° apart around the circumference of the tubing to provide two magnets equally spaced around the tubing, facing and attracted to two magnets, or two pieces of ferromagnetic material, in corresponding positions on the opposite side of the releasable magnetic connection. As another example, four magnets can be used, each 90° apart around the circumference of the tubing to provide four magnets equally spaced around the tubing on each side of the connection, facing and attracted to four magnets, or four pieces of ferromagnetic material, in corresponding positions on the opposite side of the releasable magnetic connection. Alternatively, magnets could be placed on the first side of the releasable magnetic connection, for example at positions 0° and 180° around the circumference of the tubing on one side, with pieces of ferromagnetic material disposed at positions 90° and 270° around the circumference, with the two magnets on the first side facing pieces of ferromagnetic material disposed at positions 0° and 180° around the circumference of the tubing on the second side of the releasable magnetic connection, and the two pieces of ferromagnetic material disposed at positions 90° and 270° around the circumference of the first side facing magnets disposed at positions 90° and 270° around the circumference of the second side.

In some preferred embodiments, a first magnet is formed in a configuration to match that or a second magnet, or a piece of ferromagnetic material, to be disposed on the other side of a releasable connection. Commonly, the first magnet and the corresponding second magnet or piece of ferromagnetic material are each formed in a circular form, such as a disk, of the same diameter, with a similarly sized lumen. As additional examples, the magnet may be shaped as a square or a rectangle, with a lumen surrounding the tubing of the button or exterior opening of the lumen of a button. In preferred embodiments, however, the magnet is shaped as a ring (which for purposes of this description may be a flat disc with a lumen) or a cylinder.

### Selecting Appropriate Magnetic Force

As discussed above, the force of the releasable magnetic connection is intended to be less than the force needed to dislodge the implanted medical tubing or sutured lines with which it is used. The force needed to dislodge an implanted tubing, such as a G tube, will therefore depend in part on the size and weight of the patient and in part on the retention adaptation on the tubing and is selected to be below a force that would injure the patient.

For example, a 6 pound infant has a very thin abdominal wall and a very small stomach. G tubes intended for use in such an infant have a correspondingly short section of tubing to be disposed within the infant, along with an inflation balloon sized to fit within the small stomach. Once such a G tube is implanted, a force of about 6-8 pounds may be enough to pull the G tube out. A releasable magnetic connection for a G tube for such an infant will use a magnetic force less than the weight of the child. For children less than about 10 pounds, a releasable magnetic connection of about 2 to about 4 pounds between two ends of a tube or catheter should hold the ends together during normal use but allow release if the tube or catheter is subjected to a pulling force that might otherwise dislodge the tube or catheter. For children 10-20 pounds, a releasable magnetic connection of about 3 to about 6 pounds should be acceptable. As used throughout this section, “about” means ±0.5 pounds

In contrast, a Foley catheter with its distal end implanted in the bladder of an overweight six-foot male adult may need a force of some 20-30 pounds to be pulled out of the patient. (It should be noted that Foley catheters are typically pulled out inadvertently when the external part of the tubing is caught on a door or chair as a bedridden patient is being wheeled for imaging or to the ICU and the momentum of the bed or gurney provides the force pulling out the catheter.) For endotracheal tubes, for which a common cause of unplanned extubation is arm or body movement of an agitated or cognitively impaired patient, having a releasable magnetic connection in the portion of the tubing external to the patient maintains a secure airway and is much preferable to the alternative of an unplanned extubation. For adults, a tube with a releasable magnetic connection of about 30 to 35 pounds is expected to strike a balance between reducing patient-initiated extubations and avoiding injury from force exerted on the tube. For older children and adolescents, a releasable magnetic connection of about 20 to 25 pounds is expected to strike the same balance.

IV lines sutured to the patient have somewhat different considerations. The skin of an infant is thinner than that of an adult, and there is typically less surface area over which to secure the line. In both cases, however, the force to dislodge the line is less than that needed to pull a tube or catheter with a retention device from a patient. Accordingly, it is anticipated that releasable magnetic connection of about 3 to about 5 pounds between two ends of an IV line sutured to a patient will be suitable for use with infants, about 4 to about 6 pounds for children between 1 year of age and about 7, and that connections of about 6 pounds to about 8 pounds are suitable for all other patients. It is believed that connections needing the stated amounts of force strike an appropriate balance between not having the connections disrupted unnecessarily, and preventing pain and injury to the patient by dislodgement of an implanted tube or catheter.

It is noted that doctors placing G tubes and various other tubing in patients are already accustomed to assessing patient characteristics such as weight, strength, and fragility in choosing tubes appropriate for those patients. G tubes, for example, are provided in a number of different outer diameters, denoted by the French scale or French gauge system. Practitioners routinely select the French unit of a patient's G tube based on characteristics such as size and weight of the patient. It is expected that practitioners will select, for example, a G tube with a lower strength of releasable magnetic connection for a young child, as children usually cannot protect their feeding tubes from being dislodged by caretakers who have not noticed them, and will choose a stronger magnetic connection for a feeding tube for an alert, mobile teenager, who can protect her feeding tube line. Similarly, it is anticipated that the practitioner will choose a releasable magnetic connection with a magnetic connection of about 10 pounds for an endotracheal tube to be placed in a 50 pound child, but of about 30 pounds for a normal sized adult and perhaps 40 pounds for a large or strong adult. It is anticipated that selection of an appropriate magnetic force suitable for use with respect to a particular patient and the particular retention adaptation of the particular tubing to be used for that patient can be left to the judgement of the practitioner caring for that patient.

### Attachment of Releasable Magnetic Connections to Tubing

In some embodiments, the magnets, ferromagnetic materials, or both, are attached to the medical tubing by medical grade adhesive. This is particularly convenient for tubing in which the patient is not expected to undergo an MRI with the tubing in place. Medical grade adhesives, such as epoxies and cyanoacrylates suitable for attaching metal to rubbers and plastics such as medical tubing are known in the art and are commercially available from, for example, Master Bond (Hackensack, N.J.).

Magnets or ferromagnetic materials, or both, attached to the tubing can present a concern if the patient is to undergo MRI or other procedures in which the patient is to be exposed to a high magnetic field. Practitioners caring for patients with the inventive systems also need to remain aware that the tubing contains metal if the patient needs to have an MRI, as the metal would pose a serious safety risk if exposed to the high magnetic field of a MRI. This can be dealt with in several ways. In some embodiments, such as those discussed in the preceding paragraph in which the magnets, ferromagnetic material, or both, are attached to the tubing with a medical grade adhesive, the method of attachment does not allow them to be removed from the tubing itself. In these embodiments, the tubing or portion of the tubing comprising the attached magnets, ferromagnetic material, or both, must be removed from the patient before the patient undergoes the MRI. With respect to feeding tubes in which the releasable magnetic connection is present on an extension tube connected to a button, the tube containing the connection can simply be removed before the MRI. In patients with a feeding tube system employing a button in which a magnet or ferromagnetic material is present, and not removable, the button must be removed prior to the MRI. Methods for removal of such buttons are well known. If the tract through which the tube extending distally from the button has not yet formed or is not yet well formed, conventional medical tubing can be inserted to maintain the tract during the MRI and a button with a magnet or ferromagnetic material reinserted after the desired imaging studies have been completed. Similarly, drainage tubes such as Foley tubes, and IV lines having a releasable magnetic connection in which the magnets, ferromagnetic material, or both, present in a non-removable form, must be removed prior to the MRI.

In other embodiments, the inventive systems and methods provide medical tubing in which the releasable magnetic connection has the magnets, ferromagnetic material, or both, can be removed when needed. This allows the magnet or ferromagnetic material to be removed before the MRI and to be replaced once the MRI is concluded and the patient is a safe distance away from the MRI device. The magnet or ferromagnetic material can be made removable in a variety of ways known in the art. For example, in a feeding tube system having an extension tube which connects to a button, the magnet or the ferromagnetic material, as appropriate, can be made removable by any of a number of convention methods. For example, the magnet or the ferromagnetic material, as appropriate, can be configured as a ring and placed around the first opening of the button's lumen, and while helical ridges are disposed on the outside of and surrounding the button. A plastic rim piece with helical ridges mating to those on the outside of the button can then be screwed onto the button over the magnet or ferromagnetic material, holding the magnet or ferromagnetic material in place during use but allowing them to be removed when desired. The plastic rim piece can have an open center so that the plastic rim piece does not interfere with the mating of the extension tube and the first opening of the button. If the practitioner wishes to use, say, two, three, or four magnets, or pieces of ferromagnetic material around the button rather than one ring-shaped one, the individual magnets or pieces of ferromagnetic material can be disposed around the lumen in spaces around the first opening in the button configured to receive them, and the plastic rim piece can hold them in place. In another configuration, the button can be provided with hinged prongs, allowing the prongs to snap over and hold the magnet, magnets, or ferromagnetic material in place, but which can be snapped off the magnet or ferromagnetic material when the magnets or ferromagnetic material need to be removed. In another embodiment, the magnet or ferromagnetic material can be configured to surround the lumen of a button and provided with a male helical thread mating to a female helical thread around the lumen of the button, or vice versa, allowing the magnet or ferromagnetic material to be unscrewed from the button when desired.

In some preferred in-line embodiments, the magnets, or magnet and ferromagnetic material, are attached to the line or button by a method allowing them to be removed from the line when desired. For example, the tubing can be configured with external helical ridges configured to accept mating helical ridges on the magnet or ferromagnetic material. Since magnets made of some materials, such as neodymium, are hard to machine, in other embodiments, the helical ridges on the outside of the tubing can instead mate with helical ridges on a holder holding the magnet or ferromagnetic material. For example, the magnet or ferromagnetic material can be placed, or embedded, in a holder that screws onto the helical ridges around the first side of the tubing and which holds the magnet or ferromagnetic material in position around the tubing to form the releasable magnetic connection with a magnet or ferromagnetic material on the other side of the releasable magnetic connection. In some embodiments, the magnet or ferromagnetic material on the other side of the releasable magnetic connection is also in a holder. The holder or holders are unscrewed from the tubing when desired so that the patient can undergo MRI or similar procedures. In some embodiments, one side of the releasable magnetic connection has a magnet disposed in a holder screwed onto the tubing, and the other side has a ring or cylindrical ferromagnetic material with a lumen having helical threads mating to helical threads on the tubing.

In some preferred embodiments, holders made of hard plastic or other materials compatible with the tubing hold the magnets or magnets and ferromagnetic material on the tubing in normal use, but to allow the magnets or ferromagnetic material, or both, to be removed when desired. The materials forming the holders are preferably materials approved for medical use. The holders can be configured to be part of the medical tubing, or to fit over one or both sides of the site of the releasable magnetic connection and to screw onto the tubing. The holders can be provided with external male or female helical threads, allowing magnets, or magnets and ferromagnetic material (or plastic holders containing them) with mating threads to be screwed onto the holders and to form the releasable magnetic connection, and to be unscrewed from the holders when they need to be removed. Or, the ends of the holders can flare open to provide an internal space with an inside surface, with threads on the inside surface into which magnets, ferromagnetic materials, or plastic holders containing them can be screwed, and removed when needed. Upon removal of the magnets, ferromagnetic material, or both, a separate connecting tube with threading on either end designed to mate with the holder on either side can be used as a bridge to connect the tubing across the space usually connected by the releasable magnetic connection. The connecting tube provides a lumen of medical grade to fluidly connect the tubing on either side of the connecting tube and is of materials safe for use in an MRI device. Use of the connecting tube allows the tubing to remain connected to the patient during the MRI or any other procedure in which magnets, ferromagnetic material, or both cannot be present. Holders, magnets or ferromagnetic material sized and bearing male or female threads that mate to complementary male or female threads on a button or tubing are sometimes referred to herein as “mated” helical ridges. Embodiments in which the magnets, ferromagnetic materials, or holders holding them can be firmly attached to the tubing or to a button, but removed when needed, are sometimes referred to herein as “reversibly attached.”

Placement of Magnets on Patients with Pacemakers and Other Electronic Devices

Magnets may affect the operation of some pacemakers and implantable cardioverter-defibrillator (“ICD”) and other implantable medical devices with electronic controls. Special considerations apply to patients with such devices. Pacemakers and ICDs are usually implanted under the left clavicle. If the practitioner is contemplating using a tube, catheter, or IV with a releasable magnetic connection in a patient with a pacemaker, an ICD, or a like device, the distance between the device and where the releasable magnetic connection would be located should be measured, the size and strength of the magnet(s) to be used determined, and a determination made as to the field strength to which the pacemaker or ICD would be exposed. One leading manufacturer of pacemakers has indicated that a field strength of 5 gauss or less should be safe. The website of K&M Magnetics, Inc. (www.kjmagnetics.com) has a webpage titled “Pacemaker Safety” which contains a table setting forth distances at which field strength of neodymium magnets of different sizes drops to 5 gauss. An article by Ryf et al., Technol Health Care. 2008; 16(1):13-8, indicates that neodymium magnets should be kept at least 10 inches away from pacemakers. Accordingly, practitioners contemplating use of medical tubing with a releasable magnetic connection in a patient with a pacemaker,

ICD, or other electronic device that would be affected by magnetic field, should ensure that the magnetic components of the tubing will be at least 10 inches away from the pacemaker ICD, or other electronic device both in use and while the magnetic releasable connection is in proximity to the patient.

### Adaptations to Increase Diameter of Tubing

As noted in preceding sections, medical tubing such as G tubes, Foley catheters, and other devices implanted into a space within an organ often have an adaptation so that the implanted end of the device is retained within the organ. A number of such adaptations are known in the art. For example, in some embodiments, the adaptation is an inflatable balloon. Gastrostomy tubes often include as such an adaptation an inflatable balloon which can be inflated with sterile water introduced through a balloon port provided with a separate lumen that fluidly connects the port to the balloon. In another example, Malecot® catheters have two or four wings increasing the diameter of the distal end over that of the rest of the tube, facilitating retention within an organ space. The MiniONE® Capsule Non-Balloon Button and the Capsule Dome G-Tube (Applied Medical Technologies, Inc., Brecksville, Ohio) have bolsters that, when not deployed, are disposed along the axis of the tube but which, when deployed, create a horizontal configuration increasing the diameter of the portion of the device within the organ. It is expected that persons of skill are familiar with these and other adaptations known in the art.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A medical tubing system with sections releasably held together by magnetic attraction, said system comprising
(a) a first section of medical grade elastomeric tubing having a length, an exterior, a proximal end and an opposing distal end having a diameter, a first opening at said proximal end and a second opening at said distal end, a first lumen fluidly connected to said first opening, said lumen having a diameter and extending through said first section and fluidly connecting said first opening to said second opening of said first section,
(b) a second section of medical grade elastomeric tubing having a length, a proximal end having a first diameter matching said diameter of said distal end of said first section, an exterior, a proximal end having a first opening and a distal end having a second opening, which distal end further optionally (i) has a second diameter greater than said diameter, or (ii) is adapted to expand in diameter from that of said first diameter to a larger diameter, a first lumen of said second section, said first lumen of said second section having a diameter extending through said second section and fluidly connecting said first opening to said second opening of said second section, and (c) said first and said second sections are releasably held together by a selected magnetic force between (i) at least one magnet disposed on said exterior of said distal end of said first section and at least one magnet disposed on said exterior of said proximal end of said second section, said at least one magnet on said first and second sections positioned to releasably hold said second opening of said first section and said first opening of said second section together with a first selected magnetic force, (ii) at least one magnet disposed on said exterior of said distal end of said first section and a ferromagnetic material disposed on said exterior of said proximal end of said second section, said at least one magnet and said ferromagnetic material positioned to releasably hold second opening of said first section and said first opening of said second section of tubing together with a first selected magnetic force, or (iii) a ferromagnetic material disposed on said exterior of said distal end of said first section and at least one magnet disposed on said exterior of said proximal end of said second section, said ferromagnetic material and said at least one magnet positioned to releasably hold said second opening of said first section and said first opening of said second section of together with a first selected magnetic force.

2. The medical tubing system of claim 1, further wherein
(i) said at least one magnet of element (c)(i) disposed on said distal end of said first section is configured as a ring or a cylinder around said exterior of said distal end of said first section and is attached to said exterior, and said at least one magnet of element (c)(i) disposed on said proximal end of said second section of element (c)(i) is configured as a ring or cylinder around said proximal end of said second section and is attached to said exterior,
(ii) said at least one magnet of element (c)(ii) is configured as a ring or cylinder around said exterior of said distal end of said first section and is attached to said exterior, and said ferromagnetic material magnet of element (c)(i) is configured as a ring or cylinder around said exterior of said proximal end of said second section and is attached to said exterior, or
(iii) said ferromagnetic material of element (c)(iii) is configured as a ring or cylinder around said exterior of said distal end of said first section and is attached to said exterior, and said at least one magnet of element (c)(iii) is configured as a ring or cylinder around said exterior of said proximal end of said second section and is attached to said exterior.

3. The medical tubing system of claim 1, further wherein said one least one magnet on said first section or on said second section, or both, and said ferromagnetic material, is covered with, or embedded in, a medical grade elastomeric material.

4. The medical tubing system of claim 1, further wherein said distal end of said first section terminates into a nozzle fitting into said first lumen of said second section.

5. The medical tubing system of claim 4, further wherein said nozzle is cylindrical.

6. The medical tubing system of claim 4, further wherein said nozzle has a taper.

7. The medical tubing system of claim 4, further wherein said proximal end of said second section contains a one-way valve, which valve is held open when said nozzle of said first section is present and which is in closed position when said nozzle is not present.

8. The medical tubing system of claim 1, further wherein said distal end of said second section has a diameter expanded from that of said first diameter of said second section or has an adaption to increase in diameter when desired.

9. The medical tubing system of claim 8, wherein said adaption to increase in diameter when desired is an inflatable balloon.

10. The medical tubing system of claim 8, wherein said expanded diameter of said distal end of said second section is a hemispherical dome having a wide bottom, which wide bottom is disposed facing towards said first section of said medical tubing.

11. The medical tubing system of claim 1, further wherein said second section has an external retention ring positioned between said connection of said first and second sections and said distal end of said second section.

12. The medical tubing system of claim 1, wherein said magnet, said ferromagnetic material, or both, are removable.

13. The medical tubing system of claim 12, further wherein said distal end of said first section and said proximal end of said second section are configured to reversibly hold said magnets of element (i) or said magnets and ferromagnetic materials of elements (ii) or (iii), respectively.

14. The medical tubing system of claim 13, further wherein said configuration of said ends is by (i) providing said distal end of said first section with male or female helical ridges and providing a magnet or a ferromagnetic material, or a holder holding said magnet or said ferromagnetic material with helical ridges mating to said helical ridges of said distal end of said first section, and (ii) providing said proximal end of said second section with male or female helical ridges and providing a magnet or a ferromagnetic material, or a holder holding said magnet or said ferromagnetic material with helical ridges mating to said helical ridges of said proximal end of said second section.

15. A medical tubing system with a low-profile button, said system comprising
(a) a first section of medical grade elastomeric tubing having a length, a diameter, an exterior, a proximal end and an opposing distal end, a first opening at said proximal end to allow introduction of food into said feeding tube, a first lumen fluidly connected to said first opening, said lumen having a diameter, and extending through the length of said first section from said first opening port to a second opening in said distal end of said first section,
(b) a low-profile gastrostomy button having a proximal, outward facing side and a distal side, said button further having a first opening sized to accept said distal end of said first section, and a lumen of said first opening fluidly connected to a lumen of tubing extending distally from said button to a second opening, said tubing having a first diameter, and,
(c) which said second opening in said distal end of said first section is releasably held in said first opening of said button by magnetic force created by (i) at least one magnet disposed on said exterior of said distal end of said first section and at least one magnet disposed on said exterior of said proximal, outward facing side of said button, (ii) at least one magnet disposed on said exterior of said distal end of said first section and a ferromagnetic material disposed on said exterior of said proximal, outward facing side of said button, or (iii) a ferromagnetic material disposed on said exterior of said distal end of said first section and at least one magnet disposed on said exterior of said proximal, outward facing side of said button.

16. The medical tubing system of claim 15, further wherein said distal end of said first section terminates in a nozzle which fits into said first opening of said button.

17. The medical tubing system of claim 16, further wherein said first opening of said button comprises a one-way valve, which valve is held open when said nozzle of said first section is present and which is in closed position when said nozzle is not present.

18. The medical tubing system of claim 15, further wherein said distal end of said tubing extending distally from said button has a second diameter larger than that of said first diameter of said tubing extending distally from said tubing, or has an adaption to increase to said second diameter when desired.

19. The medical tubing system of claim 18, wherein said adaption to increase to said second diameter when desired is an inflatable balloon.

20. The medical tubing system of claim 19, further wherein said button comprises an inflation port and a second lumen fluidly connected to said inflation port and to said inflatable balloon on said distal section of said tubing extending distally from said button.

21. The medical tubing system of claim 18, wherein said expanded diameter of said distal end of said tubing extending distally from said button is created by two or more wings extending from said distal end, a rim or lip of material with a diameter larger than that of the tubing extending distally from said button, an expanded tip, or a hemispherical dome having a wide bottom, which wide bottom is disposed facing towards said button.
